(19) United States
(12) Patent Application Publication
(10) Pub. No.: US 2022/0016166 A1
(43) Pub. Date: Jan. 20, 2022

(54) T-CELL EXPRESSING CHIMERIC RECEPTOR

(71) Applicant: CAFA THERAPEUTICS LIMITED, Dublin (IE)

(72) Inventors: Zonghai LI, Shanghai (CN); Zhaohui LIAO, Shanghai (CN)

(73) Assignee: CAFA THERAPEUTICS LIMITED, Dublin (IE)

(21) Appl. No.: 17/277,898

(22) PCT Filed: Sep. 23, 2019
(86) PCT No.: PCT/CN2019/107347

§ 371 Date: Oct. 4, 2021

(30) Foreign Application Priority Data
Sep. 21, 2018 (CN) 201811117875.4
Sep. 28, 2018 (CN) 201811140870.3
Aug. 29, 2019 (CN) 201910809475.8
Aug. 29, 2019 (CN) 201910809598.1

(51) Int. Cl. A61K 35/17; C07K 14/705; C07K 14/74; C07K 14/435; C07K 14/725; C07K 16/28; A61P 35/00; C12N 5/0783; C12N 15/90; C12N 9/22; C12N 15/11

(52) U.S. Cl. CPC A61K 35/17; C12N 2800/80; C07K 14/70539; C07K 14/435; C07K 14/7051; C07K 16/2878; C07K 16/2818; C07K 16/2815; C07K 16/2809; A61P 35/00; C12N 5/0636; C12N 15/907; C12N 9/22; C12N 15/11; C07K 2317/565; C07K 2317/622; C12N 2310/20; C07K 14/70578

(57) ABSTRACT

Provided is a T-cell expressing a chimeric receptor, wherein the chimeric receptor specifically recognizes BCMA, and the endogenous TCR molecule and the endogenous MHC molecule are silenced. Also provided is the use of the T-cell in the preparation of a drug for treating BCMA positive tumors.

### FIELD OF THE INVENTION

The present invention relates to the field of immune cell therapy, and specifically relates to tumor immunotherapy, especially a genetically engineered immune effector cell.

### BACKGROUND OF THE INVENTION

In recent years, tumor immunotherapy has received widespread attention. At present, the T cells used in adoptive immunotherapy are mainly derived from the patient's own body. After in vitro modification and transformation, they are returned to the patient's body. Due to the large individual differences between the patients, the therapeutic effect and safety are difficult to control, and the cost is expensive. In addition, since cell preparation requires a certain period, autologous cell therapy cannot be applied when the patient is in critical condition or the patient's own conditions are not suitable for extracting peripheral blood from the body for immune cell therapy.

Allogeneic immune effector cells refer to cells obtained from other individuals of the same species. They can not only overcome the problem of poor quality of T cell sources, and enable all patients with advanced tumors to receive immune cell therapy, but also make cell products better used in automated production, reduce production costs, and ensure product consistency. However, due to the immunogenetic differences between the donor and the recipient, after the donor's T lymphocytes entering the recipient patient's body, they will mistake the normal cells or tissues in the patient's body for the foreign targets and attack them, thereby resulting in graft versus host disease (GVHD); on the other hand, allogeneic cells as exogenous grafts may also be recognized and attacked by immune cells in the patient's body, thereby inhibiting or eliminating allogeneic cells, resulting in host versus graft response (HVGR).

In the patent PCT/EP2016/055332, Cellectis knocked out the TCR receptor and CD52 in allogeneic T cells to obtain universal CAR-T cells. Although this technology reduces the risk of GVHD, it cannot inhibit HVGR and has a certain risk in safety. In patent CN201710983276.X, Precision Biotech knocked out genes comprising TRAC, B2M and CIITA at the same time to obtain universal CAR-T cells, but there were problems such as low cell preparation efficiency.

Therefore, it is necessary to develop a chimeric receptor-modified allogeneic T cell that can reduce immune rejections of GVHD and HVGR and is not easily attacked by host NK cells.

### SUMMARY OF THE INVENTION

The purpose of the present invention is to provide a T cell expressing a chimeric receptor, which can not only inhibit the risk of GVHD and HVGR, but also is not easily attacked by NK cells.

In the first aspect of the present invention, provided is a T cell expressing a chimeric receptor that specifically recognizes BCMA, wherein the endogenous TCR molecule is silenced and the endogenous MHC molecule is silenced in the T cell.

In a specific embodiment, the “TCR molecule is silenced” refers to that the genes encoding either or both the α chain and the β chain of the TCR are silenced. In a specific embodiment, the “TCR molecule is silenced” refers to that the gene encoding the α chain of the TCR is silenced (i.e., the TRAC gene). The gene encoding the α chain of the TCR is also referred to as TRAC gene herein.

In a specific embodiment, the “TCR molecule is silenced” refers to that the gene encoding the constant region of the α chain of the TCR is silenced.

In a specific embodiment, the “TCR molecule is silenced” refers to that the first exon of the gene encoding the constant region of the α chain of the TCR is silenced.

In a specific embodiment, the “MHC molecule is silenced” refers to that a MHC class I molecule is silenced.

In a specific embodiment, the MHC molecule is an HLA molecule. Preferably, the MHC molecule is an HLA class I molecule.

Preferably, the HLA molecule is selected from at least one of molecules encoding HLA-A, HLA-B, HLA-C, B2M and CIITA. More preferably, the HLA molecule is a B2M molecule, and a gene encoding B2M herein is also referred to as a B2M gene.

In a specific embodiment, gene editing technology is used to silence the endogenous TCR molecule and the endogenous MHC molecule.

In a specific embodiment, gene editing of genes encoding the constant regions of the α and/or β chains of the TCR silences the genes encoding either or both of the α and β chains of the TCR. In a specific embodiment, gene editing is performed on the gene encoding the α chain of the TCR. Preferably, gene editing is performed on the gene encoding the constant region of the α chain of the TCR. More preferably, gene editing is performed on the first exon of the gene encoding the constant region of the α chain of the TCR.

In a specific embodiment, the MHC molecule is selected from at least one of HLA-A, HLA-B, HLA-C, B2M and CIITA molecules. In a preferred embodiment, the MHC molecule is a B2M molecule.

In a specific embodiment, gene editing of the gene encoding the constant region of the B2M silences the B2M molecule; preferably, gene editing of the first exon of the gene encoding the constant region of the B2M silences the B2M molecule.

In a specific embodiment, the gene editing technology is selected from the group consisting of: CRISPR/Cas technology, artificial Zinc Finger Nucleases (ZFN) technology, transcription activation-like effector activator-like effector (TALE) technology or TALE-CRISPR/Cas technology; preferably, CRISPR/Cas9 technology is used.

In a specific embodiment, when the B2M molecule is silenced using CRISPR/Cas9 technology, the selected gRNA sequence is as shown in SEQ ID NO:1.

In a specific embodiment, when the α chain of the TCR molecule is silenced using CRISPR/Cas9 technology, the selected gRNA sequence is as shown in SEQ ID NO: 2, 27, 28, or 29.

In a specific embodiment, the selected gRNA sequence is shown in SEQ ID NO: 1 when the B2M molecule is silenced, and the selected gRNA sequence is shown in SEQ ID NO: 2 when the α chain of the TCR molecule is silenced.

In a specific embodiment, the chimeric receptor is selected from a chimeric antigen receptor (CAR) or a T cell antigen coupler (TAC).

In a specific embodiment, the chimeric receptor is a chimeric antigen receptor (CAR).

In a specific embodiment, the chimeric antigen receptor comprises:

(i) an antibody or a fragment thereof that specifically binds to BCMA, a transmembrane region of CD28 or CD8, a costimulatory signal domain of CD28, and CD3ζ; or

(ii) an antibody or a fragment thereof that specifically binds to BCMA, a transmembrane region of CD28 or CD8, a costimulatory signal domain of CD137, and CD3ζ; or

(iii) an antibody or a fragment thereof that specifically binds to BCMA, a transmembrane region of CD28 or CD8, a costimulatory signal domain of CD28, a costimulatory signal domain of CD137, and CD3ζ.

In a specific embodiment, the chimeric receptor is TAC, comprising: (a) an extracellular domain: the extracellular domain comprises an antibody domain with an antigen-binding domain and a single-chain antibody that binds to CD3; (b) a transmembrane region; (c) an intracellular domain, which is connected to the protein kinase LCK.

In a specific embodiment, the antibody or fragment thereof that specifically binds BCMA is scFv or VHH.

In a specific embodiment, the antibody that specifically binds to BCMA comprises HCDR1 shown in SEQ ID NO: 10, HCDR2 shown in SEQ ID NO: 11, and HCDR3 shown in SEQ ID NO: 12, and LCDR1 shown in SEQ ID NO: 13, LCDR2 shown in SEQ ID NO: 14, LCDR3 shown in SEQ ID NO: 15; in a specific embodiment, the antibody has a heavy chain variable region shown in SEQ ID NO: 17 and a light chain variable region shown in SEQ ID NO: 18; in a specific embodiment, the antibody that specifically binds BCMA has the sequence shown in SEQ ID NO: 3.

In a specific embodiment, the sequence of the chimeric receptor comprises the sequence shown in SEQ ID NO: 4, 5, 6 or 7. In a specific embodiment, compared with T cell in which the endogenous TCR and the endogenous MHC are active and the chimeric receptor is expressed, the tumor cell killing ability of the T cell in vitro is not reduced.

In a specific embodiment, when the T cell is applied allogeneically, it has a low allogeneic reactivity, and is not easily rejected by NK cell.

In a specific embodiment, the T cell induces a reduced graft versus host disease (GVHD), and a reduced host versus graft response (HVGR).

In the second aspect of the present invention, provided is a use of the T cell described in the first aspect for preparing a medicament for treating a BCMA-positive tumor, in a specific embodiment, the BCMA-positive tumor is selected from multiple myeloma.

In the third aspect of the present invention, provided is a pharmaceutical composition, comprising the cell described in the first aspect and a pharmaceutically acceptable carrier or excipient.

In the fourth aspect of the present invention, provided is a kit, comprising the cell described in the first aspect or the pharmaceutical composition described in the third aspect.

### The Beneficial Effect of the Present Invention

The genetically engineered immune effector cells of the present invention target BCMA, which can effectively reduce immune rejections of GVHD and HVGR caused by allogeneic cell therapy, and is not easily attacked by NK cells.

### BRIEF DESCRIPTION OF THE DRAWINGS

FIG. 1 shows the TCR/B2M double-negative cells obtained by sorting with magnetic beads. The test results show that the CD3 and B2M-negative cells after sorting reach more than 99.6%.

FIG. 2 shows the positive rate of CAR expression. The positive rates of CAR in CAR-T cells without double gene knockout and CAR-T cells with double gene knockout of TRAC and B2M are compared. The results show the positive rate of CAR of the both are above 80%, wherein UTD refers to blank T cells, that is, uninfected T cells; BCMA CAR-T refers to normal CAR-T cells with BCMA-targeting activity, and BCMA UCAR-T refers to the double knockout universal CAR-T cells with BCMA-targeting activity.

FIG. 3 shows the killing of RPMI-8226 and NCI-H929 tumor cells in vitro. The multiple myeloma cell lines RPMI-8226 and NCI-H929 are cultured in vitro as tumor target cells, and BCMA-targeting CAR-T cells before and after double gene knockout act on the tumor cells. The results show that CAR-T cells before and after gene knockout can effectively kill RPMI-8226 and NCI-H929 tumor cells, and the killing abilities are equivalent.

FIG. 4 shows the anti-tumor effect of double gene knockout BCMA-targeting CAR-T cells in vivo.

FIG. 5 shows the in vivo survival effect of double gene knockout BCMA-targeting CAR-T cells.

FIG. 6 shows the weight changes of mice in the GVHD model. The results show that BCMA UCAR-T cells can significantly reduce the side effects caused by the GVHD reaction.

FIG. 7 shows the in vivo survival of BCMA UCAR-T cells in the GVHD model. The results show that BCMA UCAR-T cells do not cause GVHD reactions in vivo and have good safety.

FIG. 8 shows the killing ability of NK-92 cells against T cells. The results in the figure show that the lysis rate of T cells in BCMA-targeting CAR-T cells before and after double gene knockout is equivalent, indicating that The CAR-T targeting BCMA after double gene knockout does not cause abnormal rejection of NK-92 cells, and the genetically engineered immune effector cells targeting BCMA prepared in the present invention can effectively survive in patients.

### DETAIL DESCRIPTION OF THE INVENTION

After extensive and in-depth research, the inventors unexpectedly discovered that by silencing the endogenous TCR molecules and B2M molecules in T lymphocytes, allogeneic universal chimeric receptor-modified T cells can be prepared, so that it can be applied to different individuals. The present invention has been completed on this basis.

Methods and materials similar or equivalent to those described herein can be used in the practice or testing of the present invention. The publications, patent applications, patents and other references mentioned herein are all incorporated herein as reference in their entirety. In case of conflict, this specification will control. In addition, unless otherwise specified, the materials, methods, and examples are illustrative only and not intended to be limiting.

Unless otherwise specified, the practice of the present invention will use traditional techniques of cell biology, cell culture, molecular biology, transgenic biology, microbiology, recombinant DNA and immunology, which all fall within the technical scope of this field. These techniques are fully explained in the literature. See, for example, Current Protocols in Molecular Biology (Frederick M. AUSUBEL, 2000, Wileyand sonlnc, Library of Congress, USA); Molecular Cloning: A Laboratory Manual, Third Edition, (Sambrook et al., 2001, Cold Spring Harbor, NewYork: Cold Spring Harbor Laboratory Press); Oligonucleotide Synthesis (M. J. Gaited., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series. Methods In ENZYMOLOGY (J. Abelson [symbol] M. Simon, eds.-in-chief, Academic Press, Inc., New York), especially, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press. London, 1987); Hand book Of Experimental Immunology, Vols. I-IV (D. M. Weir and C. C. Blackwell, eds., 1986); and Manipulating the Mouse Embryo (Cold Spring Harbor Laboratory Press, Cold Spring Harbor, N.Y., 1986).

### The Terms

Unless specifically defined herein, all technical and scientific terms used have the same meaning as commonly understood by those skilled in the fields of gene therapy, biochemistry, genetics, and molecular biology.

T cell receptor (TCR) is a cell surface receptor that participates in T cell activation in response to antigen presentation. TCR is usually composed of two chains, α and β, which can assemble to form a heterodimer and associate with the CD3 transducing subunit to form a T cell receptor complex present on the cell surface. The α and β chains of TCR are composed of the following items: immunoglobulin-like N-terminal variable regions (V) and constant regions (C), hydrophobic transmembrane domains and short cytoplasmic regions. For immunoglobulin molecules, the variable regions of the α chain and β chain are produced by V(D)J recombination, leading to a large number of diverse antigen specificities generated in the population of T cells. However, in contrast to immunoglobulins that recognize complete antigens, T cells are activated by processed peptide fragments associated with MHC molecules, and additional dimensions are introduced into antigen recognition by T cells, which is called MHC restriction. Recognizing the MHC difference between the donor and the recipient through the T cell receptor leads to the potential development of cell proliferation and GVHD. It has been shown that the normal surface expression of TCR depends on the synergistic synthesis and assembly of all seven components of the complex (Ashwell and Klusner 1990).

The inactivation of the α chain of TCR or the f chain of TCR can lead to the elimination of TCR from the surface of T cells, thereby preventing the recognition of allogeneic antigens and the resulting GVHD.

The term “MHC” is the histocompatibility complex, which is a collective term for all the gene groups encoding the antigens of the biocompatibility complex. MHC antigens are expressed in the tissues of all higher vertebrates and are called HLA antigens in human cells, playing an important role in the response caused by transplantation. The rejection is mediated by T cells that respond to the histocompatibility antigen on the surface of the implanted tissue. MHC proteins play a vital role in T cell stimulation. Antigen-presenting cells (usually dendritic cells) display peptides belonging to degradation products of foreign proteins on the cell surface on MHC. In the presence of costimulatory signals, T cells are activated and act on target cells that also display the same peptide/MHC complex.

For example, stimulated T helper cells will target macrophages that display antigens bound to their MHC, or cytotoxic T cells (CTL) will act on virus-infected cells that display foreign viral peptides. MHC antigens are divided into MHC class I antigens and MHC class II antigens.

In humans, the HLA class I gene cluster comprises three major loci HLA-A, HLA-B, and HLA-C, as well as several minor loci. The HLA class II gene cluster also comprises three main loci: HLA-DP, HLA-DQ and HLA-DR.

The term “Human leukocyte antigen” (HLA) is the coding gene of the human major histocompatibility complex, located on chromosome 6 (6p21.31), comprising a series of closely linked loci, and is closely related to the function of the human immune system. Genes encoding HLA comprise class I, class II, and class III genes. The antigens expressed by HLA class I and class II genes are located on the cell membrane, and are MHC-I (encoded by HLA-A, HLA-B, HLA-C loci) and MHC-II (coded by HLA-D region). Class I antigens almost distribute on the surface of all cells of the body. They are heterodimers composed of heavy chains (α chain) and β2 microglobulins (B2M). Class II antigens are mainly glycoproteins locatedon the surface of macrophages and B lymphocytes.

The term “B2M” refers to β-2 microglobulin, also known as B2M, which is the light chain of MHC class I molecules. In humans, B2M is encoded by the b2m gene located on chromosome 15, opposed to other MHC genes located as gene clusters on chromosome 6. A mouse model lacking β-2 microglobulin indicates that B2M is necessary for the cell surface expression of MHC class I and the stability of the peptide binding groove. It further indicates that due to targeted mutations in the β-2 microglobulin gene, hematopoietic grafts from mice lacking normal cell surface MHC I expression were rejected by NK1.1+ cells in normal mice, indicating that the defective expression of MHC I molecules makes bone marrow cells vulnerable to rejection by the host immune system (Bix et al. 1991).

Therefore, in order to provide T cells with lower allogeneic reactivity, the T cells provided in the present invention comprise T cells that have an inactivated or mutated TCR gene and HLA gene.

The term “inactive” means that the target gene (such as the TCR gene) is no longer expressed as a functional protein.

In a specific embodiment, the method of inactivating the target gene may include deleting, frameshifting or mutating the target gene, such as introducing a rare-cutting restriction endonuclease that can break the target gene into the cell. In a specific embodiment, the cell can be transfected with a nucleic acid encoding a rare-cutting restriction endonuclease capable of breaking the gene of interest, so that the rare-cutting restriction endonuclease is expressed in the cell. The rare-cutting restriction endonuclease may be a meganuclease, a zinc finger nuclease, a CRISPR/Cas9 nuclease, an MBBBD-nuclease or a TALEN-nuclease. In a preferred embodiment, the rare-cutting restriction endonuclease is a CRISPR/Cas9 nuclease, a TALEN-nuclease.

The “inactive TCR” refers to the endogenous TCR inactivated at least one subunit gene, especially the TCRα and/or TCRβ, more preferably, the TCRα gene (also referred to as TRAC gene herein).

The “inactive MHC” means that the endogenous MHC inactivated at least one subunit gene, especially the MHC I gene, and more preferably, the B2M gene.

The term “gene editing” refers to a technique for site-directed integration of foreign genes into a certain site on the target cell genome through homologous recombination to achieve the purpose of site-directed modification and transformation of a certain gene on the chromosome. It overcomes the blindness and contingency of random integration, and is an ideal way to modify and transform biological genetic material, but its targeting efficiency is extremely low. In recent years, nuclease-guided genome targeted modification technology has developed rapidly. This type of nuclease is usually composed of a DNA recognition domain and a non-specific endonuclease domain. The recognition target site of the DNA recognition domain is used to locate the nuclease to the genomic region that needs to be edited. Then the non-specific endonuclease cuts DNA double strands, causing self-repair mechanism of DNA breaks, thereby triggering mutations in gene sequences and promoting homologous recombination. The latest developed technologies include CRISPR/Cas technology, ZFN technology, TALE technology and TALE-CRISPR/Cas technology.

In this article, “gene editing” is also referred to as “gene knockout”.

The term “molecular silencing” or “gene silencing” refers to the phenomenon that genes are not expressed or underexpressed due to various reasons without damaging the original DNA. Molecular silencing occurs at two levels, one is molecular silencing at the transcriptional level caused by DNA methylation, heterochromatinization, and position effects, and the other is post-transcriptional molecular silencing, that is, at the gene post-transcriptional level, the gene is inactivated by specific inhibition of target RNA, comprising antisense RNA, co-suppression, gene suppression, RNA interference, and microRNA-mediated translational inhibition, etc.

The term “artificial Zinc Finger Nucleases (ZFN)” technology is the first generation of nuclease site-directed modification technology. Instead of bases, zinc finger motifs that specifically recognizing the triplet DNA fragments are used as the basic units for identifying specific DNA sequences. The most classic zinc finger nuclease is formed by fusing a non-specific endonuclease FokI with a zinc finger-comprising domain, and its purpose is of course to cut a specific sequence. The repair mechanism of excision can delete the single-stranded part at the cut site of the cut DNA, and then reconnected the cut DNA together. ZFN technology has been used as the main means to induce genome mutations and to improve the efficiency of homologous recombination. However, because ZFN is affected by upstream and downstream dependent effects, the combination with target DNA sequence is unstable and cannot locate all target sequences. A lot of screening and identification work is required, and off-target mutations are prone to occur. In addition, complete sets of commercial ZFNs are expensive, cumbersome in design, and unstable in effect.

The term “transcription activator-like effector (TALE)” has DNA binding specificity, and the modular operation of the identification code is simple and convenient. The TALE-DNA binding domain is composed of tandem repeating units, most of which comprise 3 4 amino acids. The 12th and 13th amino acids of the unit are designed as repeat variable residues (RVD). TALE's RVD recognizes the 4 bases of the DNA sequence with high specificity, and the 13th amino acid directly binds specifically to the base of DNA. According to the DNA sequence, a specific TALE-DNA recognition binding domain can be constructed at any site, which can be widely used in gene sequence mutation modification and gene targeting. TALE nuclease (tanscription activator-like effector nucleases, TALENs) can be assembled by setting the DNA target sequence, assembling the TALE-DNA binding domain, and fusing the non-specific DNA cleavage domain of Fok I endonuclease. TALENs bind DNA in a targeted manner to produce DNA double-strand breaks (DSBs). DSBs activate two conserved DNA repair pathways in eukaryotic cells. Non-homologous end joining (NHEJ) can reconnect broken chromosomes. During the joining process, the broken site may introduce the loss or insertion of small fragments, thereby affecting gene function or generating gene knockout effect. If a homologous recombination vector is introduced at this time, homology-directed repair (HDR) can use a similar DNA template to guide the repair, replace the DNA sequence around the breakpoint, and achieve specific mutations or site-directed introduction of foreign DNA.

CRISPER/Cas is the third-generation gene editing technology. Compared with ZFN and TALEN technologies, it has obvious advantages. Its construction is simple and convenient, the gene editing efficiency is high when used, and the cost is low. Generally speaking, the “CRISPR system” is collectively referred to as transcripts and other elements involved in the expression of CRISPR-related (“Cas”) genes or directing their activities, comprising sequences encoding Cas genes, tracr (Trans-activating CRISPR) sequences (such as the tracrRNA or the active part of tracrRNA), tracr pairing sequence (covering “direct repeats” and partial direct repeats of tracrRNA processing in the context of endogenous CRISPR systems), guide sequences (also called “spacers” in the context of endogenous CRISPR systems”), or other sequences and transcripts from the CRISPR locus. Generally speaking, the CRISPR system is characterized by elements that promote the formation of a CRISPR complex (also referred to as a protospacer in the context of the endogenous CRISPR system) at the site of the target sequence. In the context of CRISPR complex formation, “target sequence” refers to a sequence to which the guide sequence is designed to have complementarity, wherein the hybridization between the target sequence and the guide sequence promotes the formation of the CRISPR complex. Complete complementarity is not required, provided that there is sufficient complementarity to cause hybridization and promote the formation of a CRISPR complex. A target sequence can comprise any polynucleotide, such as DNA or RNA polynucleotide. In some embodiments, the target sequence is located in the nucleus or cytoplasm of the cell. Currently, CRISPR/Cas systems are mainly divided into three types: Type 1, Type II, and Type III. The Type II CRISPR/Cas system is only found in the genome of bacteria, wherein Cas9 is a multifunctional protein with a large molecular weight, involved in the maturation of crRNAs and the subsequent interference reactions.

Generally, a guide sequence is any polynucleotide sequence that has sufficient complementarity with a target polynucleotide sequence to hybridize with the target sequence and direct the sequence-specific binding of the CRISPR complex to the target sequence. In some embodiments, when a suitable alignment algorithm is used for optimal alignment, the degree of complementarity between the guide sequence and its corresponding target sequence is about or more than about 50%, 60%, 75%, 80%, 85%, 90%, 95%, 97.5%, 99%, or more. Any suitable algorithm for aligning sequences can be used to determine the optimal alignment, non-limiting examples of which include Smith-Waterman algorithm, Needleman-Wunsch algorithm, Algorithms based on Burrows-Wheeler Transform (e.g., Burrows Wheeler Aligner), ClustalW, Clustal X, BLAT, Novoalign (Novocraft Technologies). ELAND Company (Illumina, San Diego, Calif.), SOAP (available at soap.genomics.org.cn), and Maq (available at maq.sourceforge.net).

Generally, the tracr pairing sequence includes any sequence that has sufficient complementarity with the tracr sequence to facilitate one or more of the following: (1) the excision of the guide RNA flanked with tracr pairing sequence in cells comprising the corresponding tracr sequence; and (2) the formation of a CRISPR complex at the target sequence, wherein the CRISPR complex comprises a tracr pairing sequence hybridized to the tracr sequence. Generally, the degree of complementarity is in terms of the best alignment of the tracr pairing sequence and the tracr sequence along the one with the shorter length of the two sequences. The optimal alignment can be determined by any suitable alignment algorithm, and further interpretation of the secondary structure can be made, such as self-complementarity within the tracr sequence or tracr pairing sequence. In some embodiments, when performing optimal alignment, the degree of complementarity between the tracr sequence and the tracr pairing sequence along the one with the shorter length of the two sequences is about or more than about 25%, 30%, 40%, 50%, 60%, 70%, 80%, 90%, 95%, 97.5%, 99%, or higher.

In some embodiments, the CRISPR enzyme is a part of the fusion protein comprising one or more heterologous protein domains (e.g., about or more than about 1, 2, 3, 4, 5, 6, 7, 8, 9, 10 or more domains besides the CRISPR enzyme). The CRISPR enzyme fusion protein can comprise any other proteins, and optionally a linking sequence between any two domains. Examples of protein domains that can be fused to CRISPR enzymes include, but are not limited to, epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity. Non-limiting examples of epitope tags include histidine (His) tags, V5 tags, FLAG tags, influenza virus hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT), β-galactosidase, β-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). The CRISPR enzyme can be fused to a gene sequence encoding a protein or protein fragment that binds to DNA molecules or other cellular molecules, including, but not limited to, maltose binding protein (MBP), S-tag, Lex A DNA binding domain (DBD) fusion, GAL4 DNA binding domain fusion, and herpes simplex virus (HSV) BP16 protein fusion. Additional domains that can form part of a fusion protein comprising a CRISPR enzyme are described in US 20110059502, which is incorporated herein by reference. In one embodiment, the exons of the corresponding coding genes in the constant regions of either or both of the α and β chains of the TCR are knocked out using the CRISPR/Cas technology to inactivate the endogenous TCR. Preferably, the first exon of the constant region of the α chain of the endogenous TCR is targeted to be knocked out. To “inhibit” or “suppress” the expression of the B2M or TCR means that the expression of the B2M or TCR in a cell is reduced by at least 1%, at least 5%, at least 10%, at least 20%, at least 30%, at least 40%, at least 50%, at least 60%, at least 70%, at least 80%, at least 90%, at least 95%, at least 99%, or 100%. More specifically. To “inhibit” or “suppress” the expression of B2M means that the content of the B2M in a cell is reduced by at least 1%, at least 5%, at least 10%, at least 20%, at least 30%, at least 40%, at least 50%, at least 60%, at least 70%, at least 80%, at least 90%, at least 95%, at least 99% or 100%. The expression or content of a protein in cells can be determined by any suitable method known in the art, such as ELISA, immunohistochemistry, Western Blotting or flow cytometry, using B2M or TCR specific antibodies.

The term “modification” used in the present invention refers to the change in the state or structure of the protein or polypeptide of the present invention. Modification methods can be chemical, structural and functional.

The term “T CELL ANTIGEN COUPLER (TAC)” comprises three functional domains: tumor targeting domain (comprising single-chain antibodies, designed ankyrin repeat protein (DARPin) or other targeting group 2), which is an extracellular domain, a single-chain antibody that binds to CD3 to make the TAC receptor close to other TCR receptors; the transmembrane region and the intracellular region of the CD4 co-receptor. Wherein, the intracellular region is connected to the protein kinase LCK to catalyze the phosphorylation of immunoreceptor tyrosine activation motifs (ITAMs) of the TCR complex as the initial step of T cell activation.

The term “BCMA” refers to B cell maturation antigen. In a specific embodiment, BCMA refers to human BCMA. It is a type III transmembrane protein composed of 184 amino acid residues (NCBI Reference Sequence: NP_001183.2), and the amino acid sequence is shown in SEQ ID NO: 26.

As used herein, the terms “stimulate” and “activate” are used interchangeably, and they and other grammatical forms thereof can refer to the process by which a cell changes from a resting state to an active state. The process may comprise a response to antigen, migration, and/or phenotypic or genetic changes of functional activity status. For example, the term “activation” can refer to the process of gradual activation of T cells.

For example, T cells may require at least two signals to be fully activated. The first signal can occur after the binding of TCR to the antigen-MHC complex, and the second signal can occur through the binding of costimulatory molecules (see the costimulatory molecules listed in Table 1). In vitro, anti-CD3 can simulate the first signal, and anti-CD28 can simulate the second signal. For example, engineered T cells can be activated by expressed CAR. T cell activation or T cell triggering as used herein may refer to the state of T cells that have been sufficiently stimulated to induce detectable cell proliferation, cytokine production, and/or detectable effector function.

The term “chimeric receptor” refers to a fusion molecule formed by linking DNA fragments or cDNAs corresponding to proteins from different sources using gene recombination technology, comprising an extracellular domain, a transmembrane domain and an intracellular domain. Chimeric receptors include but are not limited to: chimeric antigen receptor (CAR), modified T cell (antigen) receptor (TCR), T cell fusion protein (TFP), and T cell antigen coupler (TAC). The term “costimulatory ligand” comprises molecules on antigen-presenting cells (for example, aAPC, dendritic cells, B cells, etc.) that specifically bind to identical costimulatory molecules on T cells, thereby providing a signal, and by, for example, the first signal provided by the combination of the TCR/CD3 complex and the peptide-loaded MHC molecule jointly mediates the T cell response, including but not limited to proliferation, activation, differentiation, and the like. The costimulatory ligand may include, but is not limited to, CD7, B7-1 (CD80), B7-2 (CD86), PD-L, PD-L2, 4-1BBL, OX40L, and inducible costimulatory ligand (ICOS-L), intercellular adhesion molecule (ICAM), CD30L, CD40, CD70, CD83, HLA-G, MICA, MICB, HVEM, lymphotoxin β receptor, 3/TR6, ILT3, ILT4, HVEM, the agonist or antibody that bind the Toll ligand receptor and the ligand that specifically binds to B7-H3. Costimulatory ligands also specifically comprise antibodies that specifically bind to costimulatory molecules present on T cells, such as, but not limited to CD27, CD28, 4-1BB, OX40, CD30, CD40, PD-1, ICOS, lymphocyte function related antigen-1 (LFA-1), CD2, CD7, LIGHT, NKG2C, B7-H3 and ligands that specifically bind to CD83.

The term “costimulatory molecule” refers to an identical binding partner on a T cell that specifically binds to a costimulatory ligand, thereby mediating a costimulatory response of the T cell, such as but not limited to proliferation. Costimulatory molecules include but are not limited to MHC class I molecules, BTLAs and Toll ligand receptors.

The term “costimulatory signal” refers to a signal, by combining with cell stimulatory signal molecules, such as the TCR/CD3 combination, results in T cell proliferation and/or up- or down-regulation of key molecules.

The term “chimeric antigen receptor” or “CAR” refers to an engineered molecule that can be expressed by immune cells including but not limited to T cells. CAR is expressed in T cells and can redirect T cells to induce the killing of target cells with specificity determined by artificial receptors. The extracellular binding domain of CAR can be derived from murine, humanized or fully human monoclonal antibodies. When it is in immune effector cells, it provides the cells with specificity for target cells (usually cancer cells) and has intracellular signal generation. CAR usually comprises at least an extracellular antigen binding domain, a transmembrane domain and a cytoplasmic signaling domain (also referred to herein as “intracellular signaling domain”), which comprises functional signaling domains derived from stimulatory molecules and/or costimulatory molecules as defined below. In certain aspects, groups of polypeptides are adjacent to each other. The group of polypeptides comprises a dimerization switch that can couple polypeptides to each other in the presence of a dimerization molecule, for example, an antigen binding domain can be coupled to an intracellular signaling domain. In one aspect, the stimulatory molecule is the zeta chain that binds to the T cell receptor complex. In one aspect, the cytoplasmic signaling domain further comprises one or more functional signaling domains derived from at least one costimulatory molecule as defined below. In one aspect, the costimulatory molecule is selected from the costimulatory molecules described herein, such as 4-1BB (i.e., CD137), CD27, and/or CD28. In one aspect, the CAR comprises a chimeric fusion protein comprising an extracellular antigen binding domain, a transmembrane domain, and an intracellular signaling domain comprising a functional signaling domain derived from a stimulatory molecule. In one aspect, the CAR comprises a chimeric fusion protein comprising an extracellular antigen-binding domain, a transmembrane domain, and an intracellular signaling domain comprising a functional signaling domain derived from a costimulatory molecule. In one aspect, the CAR comprises a chimeric fusion protein comprising an extracellular antigen binding domain, a transmembrane domain, and two functional signaling domains derived from one or more costimulatory molecules.

The term “signaling domain” refers to a functional part of a protein that functions by transmitting information in a cell, and is used to regulate cell activity through a certain signaling pathway by generating a second messenger or acting as an effector in response to such a messenger.

The term “cell” and other grammatical forms thereof can refer to a cell of human or non-human animal origin. Engineered cells can also refer to cells expressing CAR.

The term “transfection” refers to the introduction of exogenous nucleic acid into eukaryotic cells. Transfection can be achieved by various means known in the art, including calcium phosphate-DNA co-precipitation, DEAE-dextran-mediated transfection, polybrene-mediated transfection, electroporation, microinjection, liposome fusion, lipofection, protoplast fusion, retroviral infection and biolistics.

The term “stable transfection” or “stably transfecting” refers to the introduction and integration of exogenous nucleic acid, DNA or RNA into the genome of the transfected cell. The term “stable transfectant” refers to a cell that stably integrates foreign DNA into the genomic DNA.

The terms “nucleic acid molecule code”, “coding DNA sequence” and “coding DNA” refer to the order or sequence of deoxyribonucleotides along a deoxyribonucleic acid chain. The order of these deoxyribonucleotides determines the order of amino acids along the polypeptide (protein) chain. Therefore, the nucleic acid sequence encodes an amino acid sequence.

The term “individual” refers to any animal, such as a mammal or a marsupial. Individuals of the present invention include, but are not limited to, humans, non-human primates (such as rhesus monkeys or other types of macaques), mice, pigs, horses, donkeys, cattle, sheep, rats, and any kind of poultry.

The term “peripheral blood mononuclear cell” (PBMC) refers to cells with mononuclear nuclei in peripheral blood, comprising lymphocytes, monocytes and the like.

The term “T cell activation” or “T cell stimulation” and other grammatically forms thereof may refer to the state of T cells that are sufficiently stimulated to induce detectable cell proliferation, cytokine production, and/or detectable effector function. In some cases, “complete T cell activation” can be similar to triggering T cell cytotoxicity. Various assays known in the art can be used to measure T cell activation. The assay can be an ELISA to measure cytokine secretion, ELISPOT, a flow cytometry assay (CD107) for measuring intracellular cytokine expression, a flow cytometry assay for measuring proliferation, and a cytotoxicity assay (51Cr release assay) for determining target cell elimination control (non-engineered cell) is usually used in the assay to be compared with an engineered cell (CAR T) to determine the relative activation of the engineered cell compared to the control. In addition, the assay can be compared with engineered cells incubated or contacted with target cells that do not express the target antigen. For example, the comparison may be a comparison with GPC3-CART cells incubated with target cells that do not express GPC3.

When used to refer to a nucleotide sequence, the term “sequence” and other grammatical forms as used herein may include DNA or RNA, and may be single-stranded or double-stranded. The nucleic acid sequence can be mutated. The nucleic acid sequence can have any length.

The term “effective amount” as used herein refers to an amount that provides a therapeutic or preventive benefit.

The term “expression vector” as used herein refers to a vector comprising a recombinant polynucleotide, which comprises an expression regulatory sequence operatively linked to the nucleotide sequence to be expressed. The expression vector comprises sufficient cis-acting elements for expression; other elements for expression can be provided by host cells or in vitro expression systems. Expression vectors include all those known in the art, such as cosmids, plasmids (e.g., naked or comprised in liposomes), and viruses (e.g., lentivirus, retrovirus, adenovirus, and adeno-associated virus).

The term “lentivirus” as used herein refers to the genus of the retroviridae family. Lentivirus is unique among retroviruses in their ability to infect non-dividing cells; they can deliver a large amount of genetic information into the DNA of host cells, so they are one of the most effective methods using gene delivery vehicles. HIV. SIV and FIV are all examples of lentiviruses. Vectors derived from lentiviruses provide a means to achieve significant levels of gene transfer in vivo.

The term “vector” as used herein is a composition that comprises an isolated nucleic acid and can be used to deliver the isolated nucleic acid into a cell. Many vectors are known in the art, including but not limited to linear polynucleotides, polynucleotides related to ionic or amphiphilic compounds, plasmids, and viruses. Therefore, the term “vector” includes autonomously replicating plasmids or viruses. The term should also be interpreted to include non-plasmid and non-viral compounds that facilitate the transfer of nucleic acids into cells, such as polylysine compounds, liposomes, and the like. Examples of viral vectors include, but are not limited to, adenovirus vectors, adeno-associated virus vectors, retroviral vectors, and the like.

As used herein, the term sequence “identity” determines the percent identity by comparing two best-matched sequences over a comparison window (for example, at least 20 positions), wherein the portion of the polynucleotide or polypeptide sequence in the comparison window may comprise additions or deletions (i.e., gaps), for example, the gaps equivalent to 20% or less of the reference sequence (which does not comprise additions or deletions) for the two sequences of the best match (e.g., 5% to 15%, or 10% to 12%). The percentage is usually calculated by determining the number of positions where the same nucleic acid base or amino acid residue occurs in the two sequences to obtain the number of correctly matched positions. The number of correctly matched positions is divided by the total number of positions in the reference sequence (i.e., window size), and multiply the result by 100 to obtain the percentage of sequence identity.

The term “exogenous” as used herein refers to a nucleic acid molecule or polypeptide that has no endogenous expression in the cell, or the expression level is insufficient to achieve the function that it has when it is overexpressed. Thus, “exogenous” includes recombinant nucleic acid molecules or polypeptides expressed in cells, such as exogenous, heterologous and overexpressed nucleic acid molecules and polypeptides.

The term “endogenous” refers to a nucleic acid molecule or polypeptide derived from a gene in the organism's own genome.

In some embodiments, the chimeric receptor of the present invention is a chimeric antigen receptor. The term “Chimeric Antigen Receptor (CAR)” as used herein refers to a tumor antigen binding domain fused to an intracellular signaling domain that can activate T cells. Frequently, the extracellular binding domain of CAR is derived from mouse or humanized or human monoclonal antibodies.

Chimeric antigen receptors usually comprise extracellular (exocellular) antigen binding regions. In some embodiments, the extracellular antigen binding region may be fully human. In other cases, the extracellular antigen binding region can be humanized. In other cases, the extracellular antigen binding region may be of murine origin, or the chimera in the extracellular antigen binding region consists of amino acid sequences from at least two different animals. In some embodiments, the extracellular antigen binding region may be non-human.

A variety of antigen binding regions can be designed. Non-limiting examples include single chain variable fragments (scFv) derived from antibodies, antigen binding regions of fragments (Fab) selected from libraries, single domain fragments, or natural ligands that bind to their homologous receptors. In some embodiments, the extracellular antigen binding region may include scFv, Fab, or natural ligands, and any derivatives thereof. The extracellular antigen binding region may refer to a molecule other than the intact antibody, which may comprise a part of the intact antibody and can bind to the antigen to which the intact antibody binds. Examples of antibody fragments may include, but are not limited to, Fv, Fab, Fab′, Fab′-SH, F(ab′)2; bifunctional antibodies, linear antibodies; single-chain antibody molecules (such as scFv); and multispecific antibodies formed from antibody fragments.

Extracellular antigen binding regions, such as scFv, Fab, or natural ligands, can be part of a CAR that determines antigen specificity. The extracellular antigen binding region can bind to any complementary target. The extracellular antigen binding region can be derived from antibodies with known variable region sequences. The extracellular antigen binding region can be obtained from antibody sequences obtained from available mouse hybridomas. Alternatively, the extracellular antigen binding region can be obtained from total extracellular cleavage sequencing of tumor cells or primary cells such as tumor infiltrating lymphocytes (TILs).

In some cases, the binding specificity of the extracellular antigen binding region can be determined by complementarity determining regions or CDRs, such as light chain CDRs or heavy chain CDRs. In many cases, the binding specificity can be determined by the light chain CDR and the heavy chain CDR. Compared with other reference antigens, the combination of a given heavy chain CDR and light chain CDR can provide a given binding pocket, which can confer greater affinity and/or specificity to the antigen (e.g., GPC3). For example, CDRs specific to glypican-3 can be expressed in the extracellular binding region of CARs, so that CARs targeting GPC3 can target T cells to GPC3-expressing tumor cells.

In certain aspects of any of the embodiments disclosed herein, the extracellular antigen binding region, such as a scFv, may comprise a light chain CDR specific for an antigen. The light chain CDR may be the complementarity determining region of the scFv light chain of an antigen binding unit such as CAR. The light chain CDR may comprise a consecutive amino acid residue sequence, or two or more consecutive amino acid residue sequences separated by non-complementarity determining regions (e.g., (such as framework regions). In some cases, a light chain CDR may comprise two or more light chain CDRs, which may be referred to as light chain CDR-1. CDR-2, and the like. In some cases, a light chain CDR may comprise three light chain CDRs, which may be referred to as light chain CDR-1, light chain CDR-2, and light chain CDR-3, respectively. In some examples, a group of CDRs present on a common light chain can be collectively referred to as light chain CDRs.

In certain aspects of any of the embodiments disclosed herein, the extracellular antigen binding region, such as a scFv, may comprise a heavy chain CDR specific for an antigen. The heavy chain CDR may be the heavy chain complementarity determining region of an antigen binding unit such as a scFv. The heavy chain CDR may comprise a consecutive amino acid residue sequence, or two or more consecutive amino acid residue sequences separated by non-complementarity determining regions (such as framework regions). In some cases, a heavy chain CDR may comprise two or more heavy chain CDRs, which may be referred to as heavy chain CDR-1, CDR-2, and the like. In some cases, the heavy chain CDR may comprise three heavy chain CDRs, which may be referred to as heavy chain CDR-1, heavy chain CDR-2, and heavy chain CDR-3, respectively. In some cases, a group of CDRs present on a common heavy chain can be collectively referred to as heavy chain CDRs.

By using genetic engineering, the extracellular antigen binding region can be modified in various ways. In some cases, the extracellular antigen binding region can be mutated so that the extracellular antigen binding region can be selected to have a higher affinity for its target. In some cases, the affinity of the extracellular antigen binding region for its target can be optimized for targets that can be expressed at low levels on normal tissues. This optimization can be done to minimize potential toxicity. In other cases, clones of extracellular antigen-binding regions with higher affinity for the membrane-bound form of the target may be superior to their soluble form counterparts. This modification can be made because different levels of targets in soluble form can also be detected, and their targeting can cause undesirable toxicity.

In some cases, the extracellular antigen binding region comprises a hinge or spacer. The terms hinge and spacer can be used interchangeably. The hinge can be considered as part of the CAR used to provide flexibility to the extracellular antigen binding region. In some cases, the hinge can be used to detect the CAR on the cell surface of the cell, especially when the antibody that detects the extracellular antigen binding region is ineffective or available. For example, the length of the hinge derived from immunoglobulin may need to be optimized, depending on the location of the epitope on the target targeted by the extracellular antigen binding region.

In some cases, the hinge may not belong to immunoglobulin, but belong to another molecule, such as the natural hinge of the CD8a molecule. The CD8a hinge may comprise cysteine and proline residues that are known to play a role in the interaction of CD8 co-receptors and MHC molecules. The cysteine and proline residues can affect the performance of the CAR. The CAR hinge can be adjustable in size. The morphology of the immune synapse between T cells and target cells also defines the distance that cannot be bridged by the CAR due to the membrane distal epitopes on the cell surface of the target molecules, i.e. using CAR with short hinge also cannot make the synapse distance to reach the approximate value that the signal can conduct. Similarly, for the membrane proximal epitopes on the antigen targeted by CAR, signal output is only observed in the context of the long hinge CAR. The hinge can be adjusted according to the extracellular antigen binding region used. The hinge can be of any length.

The transmembrane domain can anchor the CAR to the plasma membrane of a cell. The natural transmembrane portion of CD28 can be used in the CAR. In other cases, the natural transmembrane portion of CD8a can also be used in the CAR. “CD8” can be a protein that has at least 85, 90, 95, 96, 97, 98, 99, or 100% identity with NCBI reference number: NP_001759 or a fragment thereof having stimulating activity. The “CD8 nucleic acid molecule” can be a polynucleotide encoding a CD8 polypeptide. In certain cases, the transmembrane region can be the natural transmembrane portion of CD28. “CD28” may refer to a protein that has at least 85, 90, 95, 96, 97, 98, 99 or 100% identity with NCBI reference number: NP_006130 or a fragment thereof having stimulating activity. The “CD28 nucleic acid molecule” may be a polynucleotide encoding a CD28 polypeptide. In some cases, the transmembrane portion may comprise the CD8a region.

The intracellular signaling domain of the CAR may be responsible for activating at least one of the effector functions of the T cells in which the CAR has been placed. CAR can induce effector functions of T cells, for example, the effector function is cytolytic activity or auxiliary activity, comprising secretion of cytokines. Therefore, the term “intracellular signaling domain” refers to the part of a protein that transduces effector function signals and guides cells to perform specific functions. Although the entire intracellular signaling region can usually be used, in many cases it is not necessary to use the entire chain of signaling domains. In some cases, truncated portions of intracellular signaling regions are used. In some cases, the term intracellular signaling domain is therefore intended to include any truncated portion of the intracellular signaling region sufficient to transduce effector function signals.

Preferred examples of signal domains used in CAR may include T cell receptor (TCR) cytoplasmic sequences and co-receptors that act synergistically to initiate signaling after target-receptor binding, as well as any of their derivatives or variant sequences and any synthetic sequence with the same functionality of these sequences.

In some cases, the intracellular signaling domain may comprise a signal motif comprising a known immunoreceptor tyrosine activation motif (ITAM). Examples of ITAMs comprising cytoplasmic signaling sequences comprise functional signaling domains derived from proteins of TCRζ, FcRγ, FcRβ, CD3γ, CD3δ, CD3ε, CD5, CD22, CD79a, CD79b, DAP10 of CD66d, or DAP12. However, in a preferred embodiment, the intracellular signaling domain is derived from the CD3ζ chain.

An example of a T cell signaling domain comprising one or more ITAM motifs is the CD3ζ domain, also known as the T cell receptor T3ζ chain or CD247. This domain is part of the T cell receptor-CD3 complex, and plays an important role in combining the antigen recognition of several intracellular signaling pathways with the main effect activation of T cells. As used herein, CD3ζ mainly refers to human CD3ζ and its isoforms, as known from Swissprot entry P20963, comprising proteins with substantially the same sequence. As part of the chimeric antigen receptor, once again, the T3ζ chain of the whole T cell receptor is not required, and any derivative comprising the signaling domain of the T cell receptor T3ζ chain is suitable, comprising any functional equivalents thereof.

The intracellular signaling domain can be selected from any one of the domains in Table 1. In some cases, the domain can be modified so that the identity with the reference domain can be about 50% to about 100%. Any one of the domains of Table 1 can be modified so that the modified form can comprise about 50, 60, 70, 80, 90, 95, 96, 97, 98, 99 or up to about 100% identity.

The intracellular signaling region of the CAR may further comprise one or more costimulatory domains. The intracellular signaling region may comprise a single costimulatory domain, such as the zeta chain (the first-generation CAR), or the zeta chain together with CD28 or 4-1BB (the second-generation CAR). In other examples, the intracellular signaling region may comprise two costimulatory domains, such as CD28/OX40 or CD28/4-1BB (the third generation).

Together with intracellular signaling domains such as CD8, these costimulatory domains can produce downstream activation of the kinase pathway, thereby supporting gene transcription and functional cellular responses. The costimulatory domain of CAR can activate the proximal signal proteins related to CD28 (phosphatidylinositol-4,5-bisphosphate 3-kinase) or 4-1BB/OX40 (TNF-receptor-related factor adaptor protein) pathway and MAPK and Akt activation

In some cases, the signal generated by the CAR may be combined with auxiliary or costimulatory signals. For costimulatory signaling domains, the chimeric antigen receptor-like complex can be designed to comprise several possible costimulatory signaling domains. As is well known in the art, in naive T cells, T cell receptor engagement alone is not sufficient to induce the complete activation of T cells into cytotoxic T cells. The activation of intact productive T cells requires a second costimulatory signal. Several receptors that provide costimulation for T cell activation have been reported, including but not limited to CD28, OX40, CD27, CD2, CD5, ICAM-1, LFA-1 (CD11a/CD18), 4-1BBL, MyD88 and 4-1BB. The signaling pathways used by these costimulatory molecules can all act synergistically with the main T cell receptor activation signal. The signals provided by these costimulatory signaling regions can act synergistically with the main effect activation signals derived from one or more ITAM motifs (such as the CD3 zeta signaling domain), and can complete the requirement of T cell activation.

In some cases, adding costimulatory domains to chimeric antigen receptor-like complexes can enhance the efficacy and durability of engineered cells. In another embodiment, the T cell signaling domain and the costimulatory domain are fused to each other to form a signaling region.

TABLE 1
Co-stimulatory domain

| Gene marker | Abbreviations | Names |
|---|---|---|
| CD27 | CD27; T14; S152; Tp55; TNFRSF7; | CD27 molecule |
| | S152. LPFS2 | |
| CD28 | Tp44; CD28; CD28 antigen | CD28 molecule |
| TNFRSF9 | ILA; 4-1BB; CD137; CDw137 | Tumor Necrosis Factor Receptor |
| | | Superfamily Member 9 |
| TNFRSF4 | OX40; ACT35; CD134; IMD16; | Tumor Necrosis Factor Receptor |
| | TXGP1L | Superfamily Member 4 |
| TNFRSF8 | CD30; Ki-1; D1S166E | Tumor Necrosis Factor Receptor |
| | | Superfamily Member 8 |
| CD40LG | IGM; IMD3; TRAP; gp39; CD154; | CD40 Ligand |
| | CD40L; HIGM1; | |
| | T-BAM; TNFSF5; hCD40L | |
| ICOS | AILIM; CD278; CVID1 | Inducible T cell costimulator |
| ITGB2 | LAD; CD18; MF17; MFI7; LCAMB; | Integrin β2 (complement component 3 |
| | LFA-1; MAC-1 | receptor 3 and 4 subunit) |
| CD2 | T11; SRBC; LFA-2 | CD2 molecule |
| CD7 | GP40; TP41; Tp40; LEU-9 | CD7 molecule |
| KLRC2 | NKG2C; CD159c; NKG2-C | Killer cell lectin-like receptor |
| | | subfamily C, member 2 |
| TNFRSF18 | AITR; GITR; CD357; GITR-D | Tumor Necrosis Factor Receptor |
| | | Superfamily Member 18 |
| TNFRSF14 | TR2; ATAR; HVEA; HVEM; CD270; | Tumor Necrosis Factor Receptor |
| | LIGHTR | Superfamily Member 14 |
| HAVCR1 | TIM; KIM1; TIM1; CD365; HAVCR; | Hepatitis A Virus Cell Receptor 1 |
| | KIM-1; TIM-1; | |
| | TIMD1; TIMD-1; HAVCR-1 | |
| LGALS9 | HUAT; LGALS9A, Galectin-9 | Lectin, galactoside binding, soluble, |
| | | 9 |
| CD83 | BL11; HB15 | CD83 molecule |

The term “regulation” as used herein refers to a positive or negative change. Examples of regulation comprise 1%, 2%, 10%, 25%, 50%, 75%, or 100% changes.

The term “treatment” as used herein refers to clinical intervention in the process of trying to change an individual or treating a disease caused by cells. It can be used for both prevention and intervention in the clinical pathological process. The therapeutic effect includes, but is not limited to, preventing the occurrence or recurrence of the disease, reducing the symptoms, reducing the direct or indirect pathological consequences of any disease, preventing metastasis, slowing the progression of the disease, improving or relieving the condition, relieving or improving the prognosis, etc.

Genetically Engineered T Cells

The genetically engineered T cell described herein refers to a BCMA-targeting-T cell modified by means of genetic engineering, and in the genetically engineered T cell of the present invention, the TCR gene and MHC gene encoded endogenously are silenced.

According to one aspect of the present invention, the present invention also comprises a nucleic acid encoding the chimeric receptor. The present invention also relates to a variant of the above-mentioned polynucleotide, which encodes the polypeptide having the same amino acid sequence as the present invention, or fragments, analogs and derivatives thereof.

The present invention also provides a vector comprising a nucleic acid encoding the chimeric receptor protein expressed on the surface of a T cell.

In a specific embodiment, the vector used in the present invention is a lentiviral plasmid vector PRRLSIN-cPPT.EF-1α. It should be understood that other types of viral vectors and non-viral vectors are also applicable.

The present invention also comprises viruses comprising the above-mentioned vectors. The viruses of the present invention comprise viruses that have infectivity after packaging, and also comprise viruses to be packaged that comprise the necessary components for packaging infectious viruses. Other viruses known in the art that can be used to transduce foreign genes into T cells and their corresponding plasmid vectors can also be used in the present invention.

Many virus-based systems have been developed for gene transfer into mammalian cells. For example, retroviruses provide a convenient platform for gene delivery systems. The selected gene can be inserted into a vector and packaged in a retroviral particle using techniques known in the art. Vectors derived from retroviruses such as lentiviruses are suitable tools to achieve long-term gene transfer because they allow the long-term stable integration of the transgene and its propagation in daughter cells. Lentiviral vectors have additional advantages over vectors derived from retroviruses such as murine leukemia virus, because they can transduce non-proliferating cells. They also have the additional advantage of low immunogenicity. The advantage of adenoviral vectors is that they do not fuse into the genome of the target cell, thereby bypassing negative integration-related events.

Cells can be transfected with a transgene encoding the chimeric receptor. The transgene concentration can range from about 100 picograms to about 50 micrograms. In some cases, the amount of nucleic acid (e.g., ssDNA, dsDNA, or RNA) introduced into the cell can be changed to optimize the transfection efficiency and/or cell viability. For example, 1 microgram of dsDNA can be added to each cell sample for electroporation. In some cases, the amount of nucleic acid (e.g., double-stranded DNA) required for optimal transfection efficiency and/or cell viability varies depending on the cell type. In some cases, the amount of nucleic acid (e.g., dsDNA) used for each sample can directly correspond to transfection efficiency and/or cell viability, for example, a series of transfection concentrations. The transgene encoded by the vector can be integrated into the cell genome. In some cases, the transgene encoded by the vector integrates forward. In other cases, the transgene encoded by the vector integrates reverse.

In some cases, immunoreactive cells may be T stem memory TSCM cells composed of CD45RO(−), CCR7(+), CD45RA(+), CD62L+(L-selectin), CD27+, CD28+ and/or IL-7Rα+, the stem memory cells can also express CD95, IL-2R40, CXCR3 and/or LFA-1, and show many functional properties different from the stem memory cells. Alternatively, the immunoreactive cell may also be a central memory TCM cell comprising L-selectin and CCR7, wherein the central memory cell can secrete, for example, IL-2, but not IFNγ or IL-4. The immunoreactive cells can also be effector memory TEM cells comprising L-selectin or CCR7, and produce, for example, effector cytokines such as IFNγ and IL-4.

The delivery of the vector is usually by systemic administration (e.g., intravenous, intraperitoneal, intramuscular, subcutaneous, or intracranial infusion) or topical application, by administering to the individual patients in vivo, as described below. Alternatively, the vector can be delivered to cells ex vivo, such as cells removed from an individual patient (e.g., lymphocytes. T cells, bone marrow aspirate, tissue biopsy), and then the cells are usually re-implanted in the patient's body after selection of the cells incorporated with vectors. Before or after the selection, the cells can be expanded.

The T cells can be obtained from many sources, including PBMC, bone marrow, lymph node tissue, umbilical cord blood, thymus tissue, and tissue from infection sites, ascites, pleural effusion, spleen, and tumors. In certain cases, any number of techniques known to those skilled in the art, such as Ficoll™ isolation, can be used to obtain T cells from blood collected from an individual. In one embodiment, cells from the circulating blood of the individual are obtained by apheresis. Apheresis products usually comprise lymphocytes, comprising T cells, monocytes, granulocytes. B cells, other nucleated white blood cells, red blood cells and platelets. In one embodiment, the cells collected by apheresis collection can be washed to remove the plasma fraction and placed in a suitable buffer or medium for subsequent processing steps. Alternatively, cells can be derived from healthy donors, from patients diagnosed with cancer.

In some embodiments, the cell may be part of a mixed cell population with different phenotypic characteristics. It is also possible to obtain cell lines from transformed T cells according to the aforementioned method. Cells can also be obtained from cell therapy banks.

In some cases, suitable primary cells include peripheral blood mononuclear cells (PBMCs), peripheral blood lymphocytes (PBLs) and other blood cell subpopulations, such as, but not limited to T cells, natural killer cells, monocytes, natural killer T cells, monocyte precursor cells, hematopoietic stem cells or non-pluripotent stem cells. In some cases, the cell may be any T cell such as tumor infiltrating cells (TILs), such as CD3+ T cells, CD4+ T cells, CD8+ T cells, or any other type of T cells. T cells may also comprise memory T cells, memory stem T cells, or effector T cells. It is also possible to select T cells from a large population, for example from whole blood. T cells can also be expanded from large populations. T cells may also tend to a specific population and phenotype. For example, T cells can tend to have a phenotype including CD45RO(−), CCR7(+), CD45RA(+), CD62L(+), CD27(+), CD28(+) and/or IL-7Rα(+). Suitable cells can have one or more markers selected from the group consisting of that in the following list: CD45RO(−), CCR7(+), CD45RA(+), CD62L(+), CD27(+), CD28(+) and/or IL-7Rα(+). Suitable cells also include stem cells, such as, for example, embryonic stem cells, induced pluripotent stem cells, hematopoietic stem cells, neuronal stem cells, and mesenchymal stem cells. Suitable cells may comprise any number of primary cells, such as human cells, non-human cells, and/or mouse cells. Suitable cells may be progenitor cells. Suitable cells can be derived from the subject to be treated (e.g., patient).

The amount of therapeutically effective cells required in a patient can vary depending on the viability of the cells and the efficiency with which the cells are genetically modified (for example, the efficiency with which the transgene is integrated into one or more cells, or the expression level of the protein encoded by the transgene). In some cases, the cell viability result after genetic modification (e.g., doubling) and the efficiency of transgene integration may correspond to the therapeutic amount of cells available for administration to the subject. In some cases, the increase in cell viability after genetic modification may correspond to a decrease in the amount of required cells that are effective for the patient when the treatment is given. In some cases, an increase in the efficiency of integration of the transgene into one or more cells may correspond to a decrease in the number of cells required to give a therapeutically effective treatment in the patient. In some cases, determining the amount of therapeutically effective cells required can comprise determining functions related to changes in the cells over time. In some cases, determining the amount of therapeutically effective cells required can comprise determining the function corresponding to the change in the efficiency of integrating the transgene into one or more cells based on time-related variables (e.g., cell culture time, electroporation time, cell stimulation time). In some cases, a therapeutically effective cell may be a cell population that comprises about 30% to about 100% expression of chimeric receptors on the cell surface. In some cases, as measured by flow cytometry, therapeutically effective cells can express the chimeric receptor on the surface of about 30%, 35%, 40%, 45%, 50%, 55%, 60%, 65%, 70%, 75% 80%, 85%, 90%, 91%, 92%, 93%, 94%, 95%, 96%, 97%, 98%, 99%, 99.5%, 99.9% or more than about 99.9% of the cells.

Nucleic Acid

The nucleic acid described herein has the meaning conventionally understood by those skilled in the art. Specifically, the nucleic acid of the present invention refers to a nucleic acid used to transform T cells by genetic engineering means, such as the nucleic acid encoding chimeric antigen receptors, and used to inactivate the endogenous T cell receptor (TCR) and B2M of the T cells, such as used to knock out the endogenous T cell receptor (TCR) and B2M of the T cells.

Pharmaceutical Composition

The T cells of the present invention can be used to prepare a pharmaceutical composition. In addition to the effective amount of the T cells, the pharmaceutical composition may also comprise a pharmaceutically acceptable carrier. The term “pharmaceutically acceptable” means that when the molecular entities and compositions are properly administered to animals or humans, they will not produce adverse, allergic or other adverse reactions.

Specific examples of some substances that can be used as pharmaceutically acceptable carriers or components thereof are antioxidants; preservatives; pyrogen-free water; isotonic salt solutions; and phosphate buffers, and the like.

The composition of the present invention can be prepared into various dosage forms according to needs, and a physician can determine the beneficial dosage for a patient to administration according to factors such as the patient's type, age, weight, general disease condition, administration method, and the like. The method of administration can be, for example, parenteral administration (such as injection) or other treatment methods.

“Parenteral” administration of the composition includes, for example, subcutaneous (s.c.), intravenous (i.v.), intramuscular (i.m.) or intrasternal injection or infusion techniques. T cell population-containing preparation administered to an individual comprises multiple T cells effective in treating and/or preventing a specific indication or disease. Therefore, a therapeutically effective population of immunoreactive cells can be administered to the individual. Generally, a preparation comprising about 1×10⁴ to about 1×10¹⁰ immunoreactive cells is administered. In most cases, the preparation will comprise about 1×10⁵ to about 1×10⁹ immunoreactive cells, about 5×10⁵ to about 5×10⁸ immunoreactive cells, or about 1×10⁶ to about 1×10⁷ immunoreactive cells. However, depending on the location, source, identity, degree and severity of the cancer, the age and physical condition of the individual to be treated, etc., the number of CAR immunoreactive cells administered to the individual will vary within a wide range. The doctor will finally determine the appropriate dose to be used. In some embodiments, chimeric antigen receptors are used to stimulate immune cell-mediated immune responses. For example, a T cell-mediated immune response is an immune response involving T cell activation. Activated antigen-specific cytotoxic T cells can induce apoptosis in target cells displaying foreign antigen epitopes on the surface, such as cancer cells displaying tumor antigens. In another embodiment, chimeric antigen receptors are used to provide anti-tumor immunity in mammals. Due to the T cell-mediated immune response, a subject will develop anti-tumor immunity.

In some cases, the method for treating a subject with cancer may involve the administration of one or more T cells of the present invention to the subject in need of treatment. The T cells can bind tumor target molecules and induce the death of cancer cells. As described above, the present invention also provides a method for treating pathogen infection in an individual, which comprises administering to the individual a therapeutically effective amount of the T cells of the present invention.

Combination with Anti-Tumor Drugs

In some embodiments, the T cells of the present invention can be administered in combination with another therapeutic agent. In some embodiments, the other therapeutic agent is a chemotherapeutic drug. The chemotherapeutic drugs that can be used in combination with the T cells of the present invention include, but are not limited to, mitotic inhibitors (vinca alkaloids), including vincristine, vinblastine, vindesine, and Novibin™ (vinorelbine, 5′-dehydro-hydrogen sulfide); topoisomerase I inhibitors, such as camptothecin compounds, including Camptosar™ (irinotecan HCL), Hycamtin™ (topotecan HCL) and other compounds derived from camptothecin and its analogs; podophyllotoxin derivatives, such as etoposide, teniposide and mitopodozide ( [symbol] ); alkylating agents cisplatin, cyclophosphamide, nitrogen mustard, trimethylene thioxophosphamide, carmustine, busulfan, chlorambucil, belustine ( [symbol] ), uracil mustard, chlomaphazine ( [symbol] ) and dacarbazine; antimetabolites, including cytarabine, fluorouracil, methotrexate, mercaptopurine, azathioprine and procarbazine; antibiotics, including but not limited to doxorubicin, bleomycin, dactinomycin, daunorubicin, mithramycin ( [symbol] ), mitomycin, sarcomycin C, and daunorubicin; and other chemotherapeutic drugs, including but not limited to anti-tumor antibodies, dacarbazine, azacytidine, amsacrine ( [symbol] ), melphalan, ifosfamide and mitoxantrone.

In some embodiments, the chemotherapeutic drugs that can be used in combination with the T cells of the present invention include, but are not limited to, anti-angiogenic agents, including anti-VEGF antibodies (including humanized and chimeric antibodies, anti-VEGF aptamers, and antisense oligonucleotides) and other angiogenesis inhibitors, such as angiostatin, endostatin, interferon, retinoic acid, and tissue inhibitors of metalloproteinase-1 and -2.

Kit

The present invention also provides a kit comprising the T cell of the present invention. The kit can be used to treat or prevent cancer, pathogen infection, immune disorder, or allogeneic transplantation. In one embodiment, the kit may comprise a therapeutic or prophylactic composition comprising an effective amount of T cells in one or more unit dosage forms.

In some embodiments, the kit comprises a sterile container that can comprise a therapeutic or prophylactic composition.

In some cases, the kit may comprise about 1×10⁴ cells to about 1×10⁶ cells. In some cases, the kit may comprise at least about 1×10⁵ cells, at least about 1×10⁶ cells, at least about 1×10⁷ cells, at least about 4×10⁷ cells, at least about 5×10⁷ cells, at least about 6×10⁷ cells, at least about 6×10⁷ cells, 8×10⁷ cells, at least about 9×10⁸ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 6×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ Cells, at least about 6×10¹⁰ cells, at least about 9×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 8×10¹¹ cells, at least about 9×10¹¹ cells, or at least about 1×10¹² cells. For example, about 5×10¹⁰ cells can be comprised in the kit.

In some cases, the kit may comprise allogeneic cells. In some cases, the kit can comprise cells that can comprise genomic modifications. In some cases, the kit may comprise “off-the-shelf” cells. In some cases, the kit can comprise cells that can be expanded for clinical use. In some cases, the kit may comprise contents for research purposes.

Hereinafter, the preferred embodiments of the present invention will be described in detail with reference to the accompanying drawings. The examples given are to better describe the content of the present invention, but the content of the present invention is not limited to the examples. Non-essential improvements and adjustments made to the embodiments by those skilled in the art according to the above-mentioned content of the invention belong to the protection scope of the present invention.

### Example 1: Preparation of BCMA-Targeting CAR-T Cells

1. Preparation of CAR-T Cells.

a. Construction of the Chimeric Antigen Receptor Plasmid of BCMA

Using PRRLSIN-cPPT.EF-1α (purchased from Addgene) as a vector, a lentiviral plasmid PRRLSIN-cPPT.EF-1α-BCMA-BBZ expressing the second-generation chimeric antigen receptor of BCMA antibody is constructed.

BCMA-BBZ sequence consists of a CD8a signal peptide (SEQ ID NO: 19), a BCMA scFv (SEQ ID NO: 16), a CD8 hinge (SEQ ID NO: 20) and a transmembrane region (SEQ ID NO: 24), a CD137 intracellular signaling domain (SEQ ID NO: 25) and a CD34 (SEQ ID NO: 23).

b. Preparation of CAR-T Cells

The plasmid PRRLSIN-cPPT.EF-la-BCMA-BBZ is transfected into 293T cells (purchased from the Cell Bank of the Chinese Academy of Sciences) to obtain the lentivirus PRRL-BCMA-BBZ.

Peripheral blood mononuclear cells (PBMCs) are isolated from the peripheral blood of healthy people. Magnetic beads (purchased from Thermo Fisher) coupled with CD3/CD28 antibody are added to PBMCs to activate CD3-positive T cells. The T cells are culture and expand in vitro. After 48 hours of T cell activation and expansion, the cell density is adjusted, PRRL-BCMA-BBZ lentivirus are added at the ratio of MOI=4, and T cells are infected, then expand and culture to obtain chimeric antigen receptor modified BCMA CAR-T cells.

2. Knockout of TRAC and B2M Genes in CAR-T Cells.

The sequence of the first exon of TRAC (the gene encoding the α chain of TCR) is shown in SEQ ID NO: 8, and the sequence of the first exon of B2M is shown in SEQ ID NO: 9.

Obtaining TRAC gRNA: according to the gRNA sequence for TCR shown in SEQ ID NO: 2, the corresponding primers are designed, and the corresponding gRNA sequence is transcribed and amplified by the in vitro gRNA transcription kit (purchased from Thermo Fisher).

Obtaining B2M gRNA: according to the gRNA sequence for MHC shown in SEQ ID NO: 1, the corresponding primers are designed, and the corresponding gRNA sequence is transcribed and amplified by the in vitro gRNA transcription kit (purchased from Thermo Fisher).

The Cas 9 enzyme (purchased from NEB) and the gRNA of B2M as shown in SEQ ID NO: 1 and the gRNA of TCR as shown in SEQ ID NO: 2 are incubated at room temperature for 10 minutes at a ratio of 1:2 to obtain the RNP complex. CAR-T cells are mixed with RNPs, and electroporation instrument is used to introduce RNP complexes into CAR-T cells. 7 days after electroporation, flow cytometry is used to detect TRAC and B2M gene knockout situation. The knockout rates of both single gene and double genes of TRAC and B2M reach more than 70%.

In addition, the gRNA sequence of the TRAC can also be as shown in SEQ ID NO: 27, 28, or 29, which can also be used to obtain T cells with silenced TRAC.

In addition, the gRNA sequence of B2M can also be as shown in SEQ ID NO: 30, 31, or 32, which can also be used to obtain T cells with silenced B2M.

3. TCR/B2M Double Negative Cell Screening.

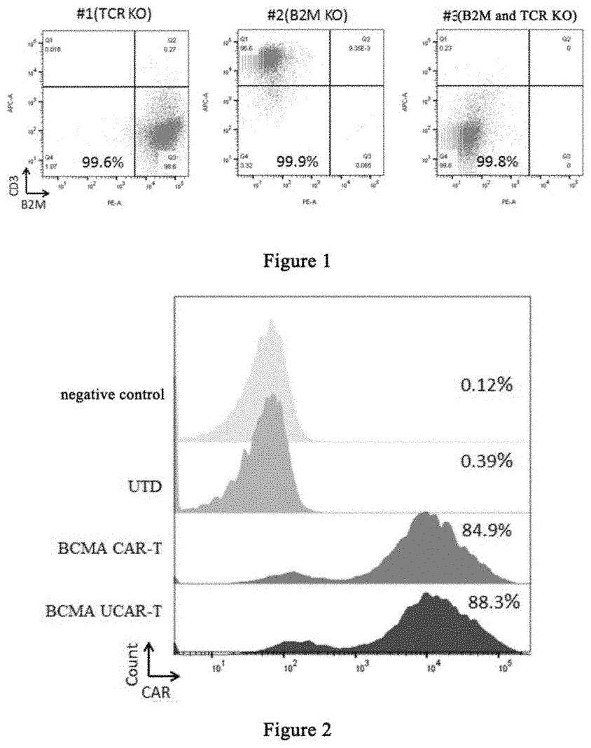

Expand B2M/TCR knockout CAR-T cells in vitro, adjust the cell density, label the cells with anti-TCR and B2M antibodies, and then label them with phycoerythrin (PE) coupled magnetic beads. After the labeled cells are sorted by a sorting column (purchased from Miltenyi), TCR and B2M negative cells are collected, and TCR and B2M double-negative universal CAR-T cells are obtained. After flow cytometry detection, the results are shown in FIG. 1. After sorting, the B2M and TCR negative cells reaches more than 99.6%.

4. Expression of CAR in Genetically Modified T Cells Targeting BCMA.

CAR-T cells without the TRAC/B2M gene knockout and TRAC/B2M double-negative universal CAR-T cells expanded in vitro are taken. T cells that are not transfected with CAR are used as a control. The cell density is adjusted. Anti-BCMA single-chain antibodies are used for labeling for the detection of CAR expression in CAR-T cells with double genes knockout. The results of flow cytometry detection are shown in FIG. 2. The positive rates of CAR in normal CAR-T cells (also referred to as BCMA CAR-T herein) and gene knocked out CAR-T cells (also referred to as BCMA UCAR-T herein) are above 80%.

### Example 2 In Vitro Tumor Killing Experiment of Double Gene Knockout BCMA-Targeting CAR-T Cells

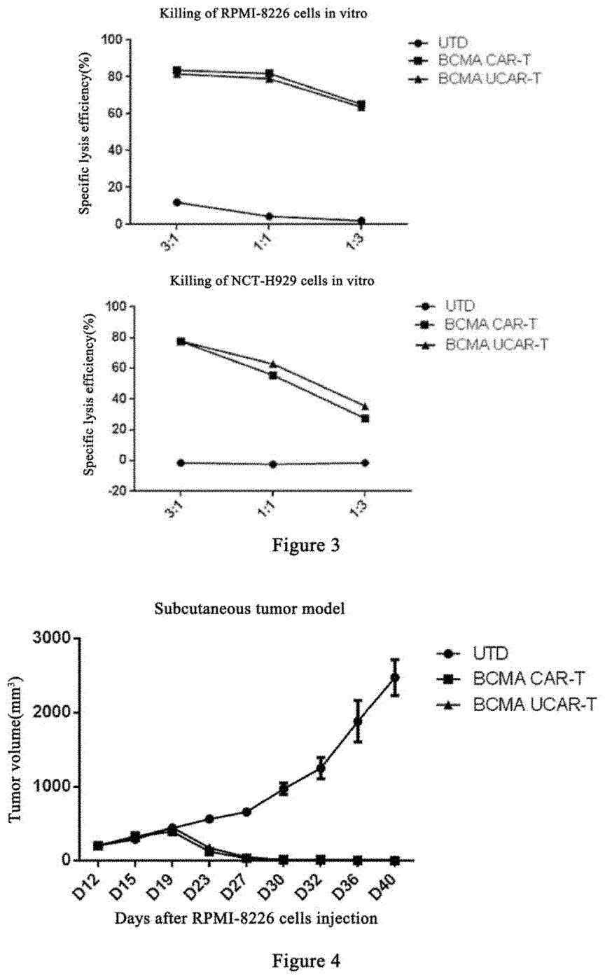

Multiple myeloma cell lines RPMI-8226 and NCI-H929 (purchased from ATCC) are cultured in vitro as target tumor cells. Adjust the cell density, inoculate a certain amount of cells into a 96-well plate. The corresponding CAR-T cells are inoculated at the three rate: effector T cells: target cells=1:3, 1:1 and 3:1. Using RPMI-1640+10% FBS as the medium, incubate them for 18 hours in a 37° C. 5% CO₂ incubator. Use cytotox-96 non-radioactive cytotoxicity assay kit (purchased from Promega), take the supernatant to determine the content of lactate dehydrogenase (LDH), and calculate the lysis efficiencies of RPMI-8226 and NCI-H929 cells in the three experimental groups of UTD. BCMA CAR-T and BCMA UCAR-T. The test results are shown in FIG. 3. Both BCMA UCAR-T and BCMA CAR-T can effectively kill RPMI-8226 and NCI-H929 cells, and the killing ability is equivalent, indicating that BCMA UCAR-T can effectively kill myeloma cells.

### Example 3 In Vivo Tumor Killing Effect of Double Gene Knockout BCMA-Targeting CAR-T Cells

The multiple myeloma cell line RPMI-8226 (purchased from ATCC) is cultured in vitro, and 5×10⁶ cells are subcutaneously inoculated into 25 NPG immunodeficient mice (purchased from Beijing Weitong Lihua Laboratory Animal Technology Co., Ltd.). The size of the tumor is measured 12 to 14 days after inoculation for the screening and grouping. The average tumor volume is about 150-200 mm³. 1×10⁶ UTD, BCMA CAR-T and BCMA UCAR-T cells are injected through tail vein respectively. After injection, body weights (comprising the day of administration in each group and euthanasia) are measured 2-3 times per week. The major and minor axes of the tumors are measured using a vernier caliper and record. The tumor volumes are calculated. Draw tumor growth curves based on the tumor volumes, and compare the differences of the tumor growth curve between the groups (tumor volume: V=½×long axis×short axis²). At the end of the experiment, the survival animals are euthanized and then the tumor tissues are stripped. The tumor weights are weighed, and the tumor weight differences between each groups are calculated. The test results are shown in FIG. 4. In the mice of BCMA UCAR-T cell and BCMA CAR-T cell groups, the tumor volumes and weights are significantly inhibited, indicating that BCMA UCAR-T cells have a good anti-tumor effect in vivo.

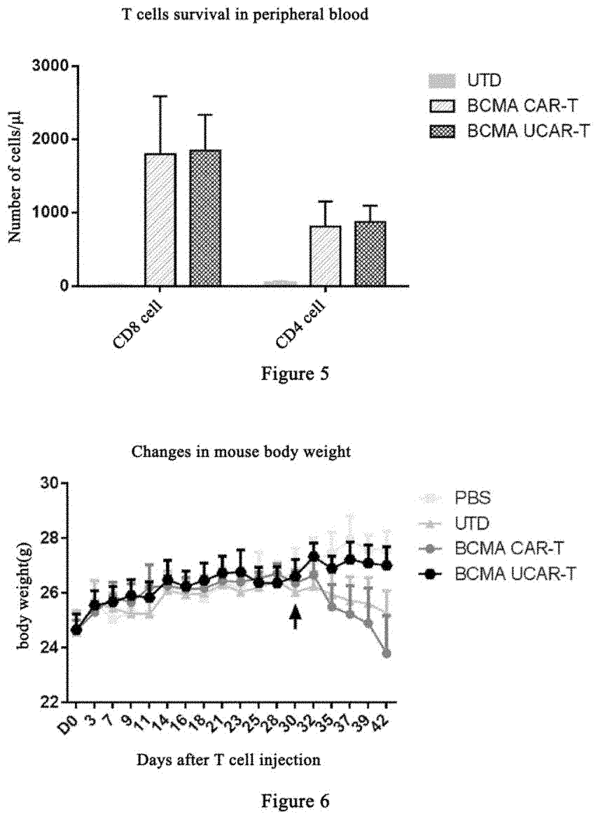

On the 11th day after the CAR-T cells injection, the peripheral blood of the mice is taken and stained with CD4 and CD8 flow cytometry antibodies, and quantified by an absolute counting tube (purchased from BD bioscience). The test results are shown in FIG. 5. In the BCMA CAR-T cell group and the BCMA UCAR-T cell group, a large number of T cells exist in the peripheral blood, indicating that BCMA UCAR-T cells are effectively expanded in mice.

### Example 4 GVHD Experiment Mediated In Vivo by Double Gene Knockout BCMA-Targeting CAR-T Cells

Use NPG immunodeficient mice to construct an in vivo GVHD model as follows.

UTD, BCMA CAR-T and BCMA UCAR-T cells are expanded and cultured in vitro, and 1×10⁷ UTD, BCMA CAR T and BCMA UCAR-T cells are injected through tail veins of NPG immunodeficient mice respectively. The negative control group is injected with saline (phosphate buffer saline. PBS). After the injection, the general clinical symptoms (physical condition, behavioral condition, death, etc.) of the mice are observed every day, the body weights are measured 2-3 times a week. Body weight growth curves based on the body weights of the mice were drawn, and the differences of body weight between the groups are compared. The results are shown in FIG. 6. From the 30th day, the body weights of the mice in groups injected with the UTD and BCMA CAR-T are decreased significantly, while the body weights of the mice in groups injected with the BCMA UCAR-T and the PBS are maintained at a normal level, indicating that the BCMA UCAR-T cells can significantly reduce the side effects caused by the GVHD reaction.

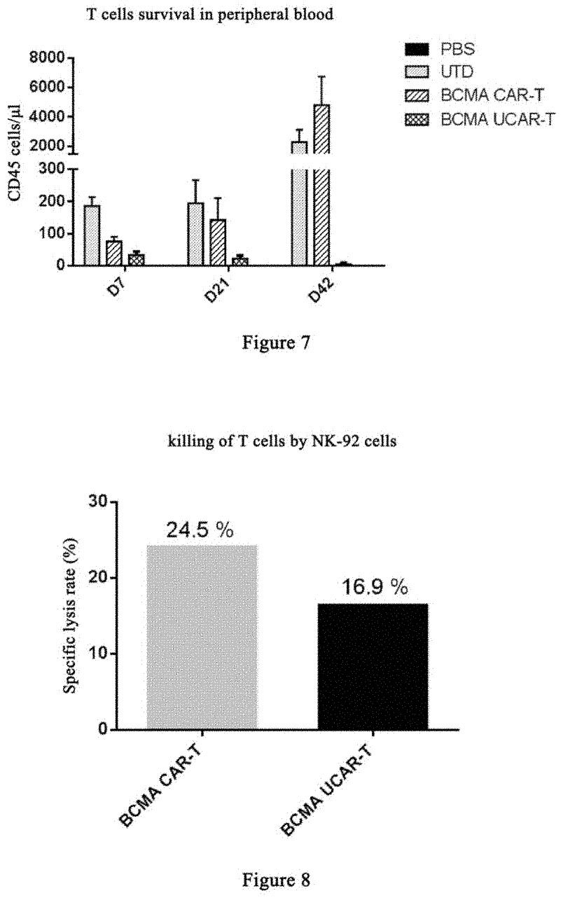

At the same time, on the 7th, 21st, and 42nd day after T cell injection, the peripheral blood of the mice is taken for flow cytometry, and the survival of T cells in the peripheral blood of each group is compared. The results are shown in FIG. 7. As time progresses, the numbers of T cells in the peripheral blood of the UTD and BCMA CAR-T groups continue to increase, while the number of T cells in the BCMA UCAR-T group gradually decreases, indicating that the BCMA UCAR-T cells can significantly reduce the abnormal proliferation of T cells caused by the GVHD response.

The above experimental results indicate that BCMA UCAR-T cells will not cause GVHD reactions in vivo and have good safety.

### Example 5 Rejection of Allogeneic NK-92 Cells to Double Gene Knockout CAR-T Cells

Studies have shown that knocking out MHC molecules (such as the B2M gene in the present invention) on normal cells may enhance the killing effect of NK cells on B2M-deficient cells. Therefore, we use the NK-92 cell line to detect its rejection effect on BCMA UCAR-T cells. Adjust the concentration of T cells and inoculate them to a 96-well plate. Inoculate NK-92 cells of the same volume and number at the ratio that NK-92 cells to T cells is 1:1. Cells are incubated in an incubator for 24 hours. Take the supernatant to determine the content of lactate dehydrogenase (LDH) and calculate the lysis efficiency of T cells. The test results are shown in FIG. 8. The lysis levels of BCMA UCAR-T and BCMA CAR-T are equivalent, indicating that the BCMA-targeting double-gene knockout CAR-T does not cause abnormal rejection by NK-92 cells.

The sequences used in the present invention are shown in the following table:

| Seq | | |
|---|---|---|
| ID | | |
| NO. | name | sequence |
| 1 | grna-1 of | TATAAGTGGAGGCGTCGCGC |
| | b2m | |
| 2 | grna-1 of | GAGAATCAAAATCGGTGAAT |
| | trac | |
| 3 | bcma | EVQLLESGGGLVQPGGSLRLSCAASGFTFGGNAMSWVRQAPGKGL |
| | antibody | EWVSAISGNGGSTFYADSVKGRFTISRDNSKNTLYLQMNSLRAEDT |
| | | AVYYCAKVRPFWGTFDYWGQGTLVTVSSGGGGSGGGGSGGGGSE |
| | | PVLTQSPGTLSLSPGERATLSCPASQSVSSSYLAWYQQKPGQAPRLL |
| | | IYGASSRATGIPDRFSGSGSGTDFTLTISRLEPEDFAVYNCQQYFNPP |
| | | EYTFGQGTKVEIKR |
| 4 | car-bcma-c | EVQLLESGGGINQPGGSLRLSCAASGFITGGNAMSWVRQAPGKGL |
| | d3z | EWVSAISGNGGSTFYADSVKGRFTISRDNSKNTLYLQMNSLRAEDT |
| | | AVYYCAKVRPFWGTFDYWGQGTLVTVSSGGGGSGGGGSGGGGSE |
| | | IVLTQSPGTLSLSPGERATLSCRASQSVSSSYLAWYQQKPGQAPRLL |
| | | IYGASSRATGIPDRFSGSGSGTDFTLTISRLEPEDFAVYYCQQYFNPP |
| | | EYTFGQGTKVEIKRRVKFSRSADAPAYQQGQNQLYNELNLGRREE |
| | | YDVLDKRRGRDPEMGGKPQRRKNPQEGLYNELQKDKMAEAYSEI |
| | | GMKGERRRGKGHDGLYQGLSTATKDTYDALHMQALPPR |
| 5 | car-bcma-2 | EVQLLESGGGLVQPGGSLRLSCAASGFTFGGNAMSWVRQAPGKGL |
| | 8z | EWVSAISGNGGSTFYADSVKGRFTISRDNSKNTLYLQMNSLRAEDT |
| | | AVYYCAKVRPFWGTFDYWGQGTLVTVSSGGGGSGGGGSGGGGSE |
| | | PVLTQSPGTLSLSPGERATLSCPASQSVSSSYLAWYQQKPGQAPRLL |
| | | IYGASSRATGIPDRFSGSGSGTDFTLTISRLEPEDFAVYNCQQYFNPP |
| | | EYTFGQGTKVEIKRTITPAPRPPTPAPTIASQPLSLRPEACRPAAGG |
| | | AVHTRGLDFACDFWVLVVVGGVLACYSLINTVAFIIFWVRSKRSR |
| | | LLHSDYMNMTPRRPGPTRKKHYQPYAPPRDFAAYRSRVKFSRSADA |
| | | PAYQQGQNQINNELNLGRREEYDVLDKRRGRDPEMGGKPQRRKN |
| | | PQEGLYNELQKDKMAEAYSEIGMKGERRRGKGHDGLYQGLSTAT |
| | | KDTYDALFIMQALPPR |
| 6 | car-bcma-b | EVQLLESGGGINQPGGSLRLSCAASGFTFGGNAMSWVRQAPGKGL |
| | bz | EWVSAISGNGGSTFYADSVKGRPTISRDNSKNTLYLQMNSLRAEDT |
| | | AVYYCAKVRPFWGTFDYWGQGTLVTVSSGGGGSGGGGSGGGGSE |
| | | IVLTQSPGTLSLSPGERATLSCRASQSVSSSYLAWYQQKPGQAPRLL |
| | | IYGASSRATGIPDRFSGSGSGTDFTLTISRLEPEDFAVYYCQQYFNPP |
| | | EWEGQGTKVEIKRITTPAPRPPITAPTIASQPLSLRPEACRPAAGG |
| | | AVHTRGLDFACDIYIWAPLAGTCGVLLLSLVITLYCKRGRKKLLYI |
| | | FKQPEMRPVQTTQEEDGCSCRFPEEEEGGCELRVKFSRSADAPAYK |
| | | QGQNQLYNELNLGRREEYDVIDKRRGRDPEMGGKPRRKNPQEGL |
| | | YNELQKDKMAEAYSEIGMKGERRRGKGHDGLYQGLSTATKDTYD |
| | | ALHMQALPPR |
| 7 | car-bcma-2 | EVQLLESGCBGLVQPGGSLRLSCAASGFTFGGNAMSWVRQAPGKGL |
| | 8bbz | EWVSAISGNGGSTFYADSVKGRFTISRDNSKNTLYLQMNSLRAkEDT |
| | | AVYYCAKVRPFWGTFDYWGQGTLVTVSSGGGGSGGGGSGGGGSE |
| | | IVLTQSPGTLSLSPGERATLSCPASQSVSSSYLAWYQQKPGQAPRLL |
| | | IYGASSRATGIPDRFSGSGSGTDFTLTISRLEPEDFAVYYCQQYFNPP |
| | | EYTFGQGTKVEIKRTTTPAPRPPTPAPTIASQPLSLRPEACRPAAGG |
| | | AVHTRGLDFACDFWVLVVVGGVLACYSLLVTVAFIIFWVRSKRSR |
| | | LLHSDYMNMTPRRPGPTRKHYQPYAPPRDFAAYRSKRGRKKLLYI |
| | | FKQPFMRPVQTTQEEDGCSCRFPEEEEGGCELRVKFSRSADAPAYQ |
| | | QGQNQINNELNLGRREEYDVLDKRRGRDPEMGGKPQRRKNNEG |
| | | LYNELQKDKMAEAYSEIGMKGERRRGKGHDGLYQGLSTATKDTY |
| | | DALHMQALPPR |
| 8 | sequence of | atatccagaaccctgaccctgccgtgtaccagctgagagactctaaatccagtgacaagtctgtctgcctatt |
| | trac-exon 1 | caccgattttgattctcaaacaaatgtgtcacaaagtaaggattctgatgtgtatatcacagacaaaactgtgc |
| | | tagacatgaggtctatggacttcaagagcaacagtgctgtggcctggagcaacaaatctgactagcatgtg |
| | | caaacgccttcaacaacanattattccagaagacaccttcccccagcccagg |
| 9 | b2m-exon 1 | aatataagtggaggcgtcgcgctggcgggcattcctgaagctgacagcattcgggccgagatgtctcgct |
| | | cctgtggccttagctgtgctcacgctactctctctttctggcctggaggctatccagc |
| 10 | hcdr1 of | GNAMS |
| | bcma | |
| | antibody | |
| 11 | hcdr2 of | AISGNGGSTFYADSVKG |
| | bcma | |
| | antibody | |
| 12 | hcdr3 of | VRPFWGTFDY |
| | bcma | |
| | antibody | |
| 13 | lcdr1 of | RASQSVSSSYLA |
| | bcma | |
| | antibody | |
| 14 | lcdr2 of | GASSRAT |
| | bcma | |
| | antibody | |
| 15 | lcdr3 of | QQYFNPPEY |
| | bcma | |
| | antibody | |

All the documents mentioned in the present invention are cited as references in this application, as if each document is individually cited as a reference. In addition, it should be understood that after reading the above teaching content of the present invention, those skilled in the art can make various changes or modifications to the present invention, and these equivalent forms also fall within the scope defined by the appended claims of the present application.

1-22. (canceled)

23. A T cell expressing a chimeric receptor that specifically recognizes BCMA, wherein the endogenous TCR molecule is silenced and the endogenous MHC molecule is silenced in the T cell.

24. The T cell of claim 23, wherein the “TCR molecule is silenced” refers to that the genes encoding either or both the α chain and the β chain of the TCR are silenced;
preferably, the “TCR molecule is silenced” refers to that the gene encoding the α chain of the TCR is silenced (i.e., the TRAC gene);
more preferably, the “TCR molecule is silenced” refers to that the gene encoding the constant region of the α chain of the TCR is silenced;
further preferably, the “TCR molecule is silenced” refers to that the first exon of the gene encoding the constant region of the α chain of the TCR is silenced.

25. The T cell of claim 23, wherein the MHC molecule is an HLA molecule.

26. The T cell of claim 23, wherein the HLA molecule is selected from HLA-I and/or HLA-II, comprising at least one of HLA-A, HLA-B, HLA-C, B2M and CIITA molecules.

27. The T cell of claim 26, wherein the HLA molecule is an HLA class I molecule, preferably the HLA molecule is a B2M molecule.

28. The T cell of claim 23, wherein gene editing technology is used to silence the endogenous TCR molecule and the endogenous MHC molecule.

29. The T cell of claim 28, wherein the gene editing technology is selected from the group consisting of: CRISPR/Cas technology, artificial Zinc Finger Nucleases (ZFN) technology, transcription activation-like effector activator-like effector (TALE) technology or TALE-CRISPR/Cas technology; preferably, CRISPR/Cas technology is used; more preferably, CRISPR/Cas9 technology is used.

30. The T cell of claim 29, wherein when the B2M molecule is silenced using CRISPR/Cas9 technology, the selected gRNA sequence is as shown in SEQ ID NO:1.

31. The T cell of claim 29, wherein when the α chain of the TCR molecule is silenced using CRISPR/Cas9 technology, the selected gRNA sequence is as shown in SEQ ID NO: 2, 27, 28, or 29.

32. The T cell of claim 30, wherein the selected gRNA sequence is shown in SEQ ID NO: 1 when the B2M molecule is silenced, and the selected gRNA sequence is shown in SEQ ID NO: 2 when the α chain of the TCR molecule is silenced.

33. The T cell of claim 23, wherein the chimeric receptor is selected from a chimeric antigen receptor (CAR) or a T cell antigen coupler (TAC).

34. The T cell of claim 33, wherein the chimeric antigen receptor comprises:
(i) an antibody or a fragment thereof that specifically binds to BCMA, a transmembrane region of CD28 or CD8, a costimulatory signal domain of CD28, and CD3ζ; or
(ii) an antibody or a fragment thereof that specifically binds to BCMA, a transmembrane region of CD28 or CD8, a costimulatory signal domain of CD137, and CD3ζ; or
(iii) an antibody or a fragment thereof that specifically binds to BCMA, a transmembrane region of CD28 or CD8, a costimulatory signal domain of CD28, a costimulatory signal domain of CD137, and CD3ζ.

35. The T cell of claim 34, wherein the antibody or fragment thereof that specifically binds BCMA comprises Fv, Fab, Fab′, Fab′-SH, F(ab′)2; a bifunctional antibody, a linear antibody; a single-chain antibody molecule (such as scFv); or a multispecific antibody formed by antibody fragments.

36. The T cell of claim 34, wherein the antibody that specifically binds to BCMA comprises HCDR1 shown in SEQ ID NO: 10, HCDR2 shown in SEQ ID NO: 11, and HCDR3 shown in SEQ ID NO: 12, and LCDR1 shown in SEQ ID NO: 13, LCDR2 shown in SEQ ID NO: 14, LCDR3 shown in SEQ ID NO: 15;
preferably, the antibody has a heavy chain variable region shown in SEQ ID NO: 17 and a light chain variable region shown in SEQ ID NO: 18; more preferably, the antibody that specifically binds BCMA has the sequence shown in SEQ ID NO: 3.

37. The T cell of claim 36, wherein the sequence of the chimeric receptor comprises the sequence shown in SEQ ID NO: 4, 5, 6 or 7.

38. The T cell of claim 23, wherein, compared with T cell in which the endogenous TCR molecule is not silenced and the endogenous MHC molecule is not silenced and the chimeric receptor is expressed, the tumor cell killing ability of the T cell in vitro is not reduced.

39. The T cell of claim 23, wherein when the T cell is applied allogeneically, it has a low allogeneic reactivity, preferably it induces a reduced graft versus host disease (GVHD) and a reduced host versus graft response (HVGR), it is further preferred that the T cell is not easily rejected by NK cell.

40. The T cell of claim 23, wherein the T cell is obtained from the following sources, comprising: PBMC, bone marrow, lymph node tissue, cord blood, thymus tissue, tissue from an infected site, ascites, pleural effusion, spleen tissue or tumor tissue.

41. Use of the T cell of claim 23 for preparing a medicament for treating a BCMA-positive tumor; preferably, the BCMA-positive tumor is selected from multiple myeloma.

42. A pharmaceutical composition, comprising the cell of claim 23 and a pharmaceutically acceptable carrier or excipient.

43. A kit, comprising the T cell of claim 23.

44. The T cell of claim 31, wherein the selected gRNA sequence is shown in SEQ ID NO: 1 when the B2M molecule is silenced, and the selected gRNA sequence is shown in SEQ ID NO: 2 when the α chain of the TCR molecule is silenced.

45. A kit, comprising the pharmaceutical composition of claim 42.


SEQUENCE LISTING (32 sequences)

SEQ ID NO: 1 - DNA, 20 bp, Artificial Sequence
  Synthetic sequence
tataagtggaggcgtcgcgc

SEQ ID NO: 2 - DNA, 20 bp, Artificial Sequence
  Synthetic sequence
gagaatcaaaatcggtgaat

SEQ ID NO: 3 - protein, 244 aa, Artificial Sequence
  Synthetic sequence
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 4 - protein, 357 aa, Artificial Sequence
  Synthetic sequence
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 5 - protein, 470 aa, Artificial Sequence
  Synthetic sequence
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 467 aa, Artificial Sequence
  Synthetic sequence
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 512 aa, Artificial Sequence
  Synthetic sequence
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - DNA, 274 bp, Artificial Sequence
  Synthetic sequence
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagg

SEQ ID NO: 9 - DNA, 127 bp, Artificial Sequence
  Synthetic sequence
aatataagtggaggcgtcgcgctggcgggcattcctgaagctgacagcattcgggccgagatgtctcgctccgtggccttagctgtgctcgcgctactctctctttctggcctggaggctatccagc

SEQ ID NO: 10 - protein, 5 aa, Artificial Sequence
  Synthetic sequence
GNAMS

SEQ ID NO: 11 - protein, 17 aa, Artificial Sequence
  Synthetic sequence
AISGNGGSTFYADSVKG

SEQ ID NO: 12 - protein, 10 aa, Artificial Sequence
  Synthetic sequence
VRPFWGTFDY

SEQ ID NO: 13 - protein, 12 aa, Artificial Sequence
  Synthetic sequence
RASQSVSSSYLA

SEQ ID NO: 14 - protein, 7 aa, Artificial Sequence
  Synthetic sequence
GASSRAT

SEQ ID NO: 15 - protein, 9 aa, Artificial Sequence
  Synthetic sequence
QQYFNPPEY

SEQ ID NO: 16 - DNA, 732 bp, Artificial Sequence
  Synthetic sequence
gaggtgcaattgctggagtctgggggaggcttggtacagcctggggggtccctgagactctcctgtgcagcctccggattcacctttggcggtaatgccatgtcctgggtccgccaggctccagggaaggggctggagtgggtctcagcaattagtggtaatggtggtagtacattctacgcagactccgtgaagggccggttcaccatctccagagacaattccaagaacacgctgtatctgcagatgaacagcctgagagccgaggacacggccgtatattactgtgcgaaagttcgtccattctggggtactttcgactactggggccaaggaaccctggtcaccgtctcgagtggtggaggcggttcaggcggaggtggttctggcggtggcggatcggaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtacttcaacccaccagaatacacgttcggccaggggaccaaagtggaaatcaaacgt

SEQ ID NO: 17 - protein, 119 aa, Artificial Sequence
  Synthetic sequence
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSS

SEQ ID NO: 18 - protein, 109 aa, Artificial Sequence
  Synthetic sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIK

SEQ ID NO: 19 - DNA, 63 bp, Artificial Sequence
  Synthetic sequence
atggccttaccagtgaccgccttgctcctgccgctggccttgctgctccacgccgccaggccg

SEQ ID NO: 20 - DNA, 135 bp, Artificial Sequence
  Synthetic sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 21 - DNA, 81 bp, Artificial Sequence
  Synthetic sequence
ttttgggtgctggtggtggttggtggagtcctggcttgctatagcttgctagtaacagtggcctttattattttctgggtg

SEQ ID NO: 22 - DNA, 123 bp, Artificial Sequence
  Synthetic sequence
aggagtaagaggagcaggctcctgcacagtgactacatgaacatgactccccgccgccccgggccaacccgcaagcattaccagccctatgccccaccacgcgacttcgcagcctatcgctcc

SEQ ID NO: 23 - DNA, 339 bp, Artificial Sequence
  Synthetic sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 24 - DNA, 63 bp, Artificial Sequence
  Synthetic sequence
atctacatctgggcgcccttggccgggacttgtggggtccttctcctgtcactggttatcacc

SEQ ID NO: 25 - DNA, 126 bp, Artificial Sequence
  Synthetic sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 26 - protein, 184 aa, Artificial Sequence
  Synthetic sequence
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 27 - DNA, 20 bp, Artificial Sequence
  Synthetic sequence
agagtctctcagctggtaca

SEQ ID NO: 28 - DNA, 20 bp, Artificial Sequence
  Synthetic sequence
tctctcagctggtacacggc

SEQ ID NO: 29 - DNA, 20 bp, Artificial Sequence
  Synthetic sequence
ctctcagctggtacacggca

SEQ ID NO: 30 - DNA, 20 bp, Artificial Sequence
  Synthetic sequence
ggccacggagcgagacatct

SEQ ID NO: 31 - DNA, 20 bp, Artificial Sequence
  Synthetic sequence
ggccgagatgtctcgctccg

SEQ ID NO: 32 - DNA, 20 bp, Artificial Sequence
  Synthetic sequence
gagtagcgcgagcacagcta